(19) United States
(12) Patent Application Publication
(10) Pub. No.: US 2009/0222044 A1
(43) Pub. Date: Sep. 3, 2009

(54) Minimally Invasive Retractor Screw and Methods of Use

(75) Inventor: Josef Gorek, Ross, CA (US)

Correspondence Address: CARTER, DELUCA, FARRELL & SCHMIDT, LLP, 445 BROAD HOLLOW ROAD, SUITE 420, MELVILLE, NY, 11747, US

(73) Assignee: K2M, Inc., Leesburg, VA (US)

(21) Appl. No.: 12/395,970

(22) Filed: Mar. 2, 2009

Related U.S. Application Data
(60) Provisional application No. 61/032,160, filed on Feb. 28, 2008.

(51) Int. Cl. A61B 17/88; A61B 17/70; A61B 1/32

(52) U.S. Cl. 606/279; 606/104; 600/219

(57) ABSTRACT

A device, system and method for orthopedic spine surgery using a novel screw-based retractor, disclosed herein, that allows for access to the spine through a minimally or less invasive approach. The retractor device is designed as an integrally formed part of the tulip of a pedicle screw assembly with opposed arms of the retractor spread apart to open the wound proximally. The arms are removed by separating the arms from the tulip and pulling it out of the wound. The retractor device is intended to be made of a stiff material, sterile packaged and disposable after one use. A system and method for using the retractor and performing a minimally invasive spine surgical procedure are also disclosed.

### CROSS-REFERENCE TO RELATED APPLICATIONS

This application is claims the benefit of, and priority to, U.S. Provisional Patent Application No. 60/032,160, filed Feb. 28, 2008, the entire contents of which are incorporated by reference herein.

### BACKGROUND

1. Technical Field

The present disclosure relates generally to orthopaedic spine surgery and in particular to a minimally invasive retractor and methods for use in a minimally invasive surgical procedure.

2. Background of the Technology

There has been considerable advancement in the development of retractors and retractor systems that are adapted for use in less invasive procedures. Many of the recent developments are based on traditional types of surgical retractors for open procedures, predominantly table-mounted devices of various designs. These devices tend to be cumbersome and are not well adapted for use in small incisions. Standard hand-held surgical retractors are well known in the prior art and can be modified to fit the contours of these small incisions, but they require manual manipulation to maintain a desired placement, thereby occupying one hand of the physician or requiring another person to assist the physician during the procedure. Typical retractors are also positioned into the soft tissue and are levered back to hold the wound open, frequently requiring re-positioning if they dislodge, obstruct the physician's view, or interfere with access to the surgical site.

In recent years, minimally invasive surgical approaches have been applied to orthopedic surgery and more recently to spine surgery, such as instrumented fusions involving one or more vertebral bodies. Unlike minimally invasive procedures such as arthroscopic knee surgery or gallbladder surgery where the affected area is contained within a small region of the body, spinal fusion surgery typically encompasses a considerably larger region of the patient's body. In addition, arthroscopic surgery and laparoscopic surgery permit the introduction of fluid (i.e. liquid or gas) for distending tissue and creating working space for the surgeon. Surgery on the spine does not involve a capsule or space that can be so distended, instead involving multiple layers of soft tissue, bone, ligaments, and nerves. For these reasons, the idea of performing a minimally invasive procedure on the spine has only recently been approached.

By way of example, in a typical spine fusion at least two vertebral bodies are rigidly connected using screws implanted into the respective vertebral bodies with a solid metal rod spanning the distance between the screws. This procedure is not generally conducive to a minimally invasive approach. The insertion of pedicle or facet screws is relatively straightforward and can be accomplished through a minimal incision. The difficulty arises upon the introduction of a length of rod into a very small incision with extremely limited access and visibility. A single level fusion may require a 30-40 mm rod to be introduced into a 1 cm incision and a multilevel fusion may require a rod several inches long to fit into a 1 cm incision. For this reason, it is important that the minimal incision be maintained in an open and accessible condition (i.e. as wide as practicable) for introduction of the rod.

Minimally invasive surgery offers significant advantages over conventional open surgery. First, the skin incision and subsequent scar are significantly smaller. By using more than one small incision rather than one large incision, the need for extensive tissue and muscle retraction may be greatly reduced. This leads to significantly reduced post-operative pain, a shorter hospital stay, and a faster overall recovery.

Most spine implant procedures are open procedures, and while many manufacturers advertise a minimally invasive method, the procedure is typically not recommended for fusions and focuses on more common and accepted minimally invasive spine procedures such as kyphoplasty, vertebroplasty, and discectomy.

Medtronic Sofamor Danek's SEXTANT® is a minimally invasive device used for screw and rod insertion. Its shortcomings lie with how complicated the system is to use and the requirement for an additional incision for rod introduction. This system also requires that the guidance devices be rigidly fixed to the pedicle screw head in order to maintain instrument alignment and to prevent cross-threading of the setscrew. For these reasons, the surgeon cannot access the surrounding anatomy for complete preparation of the field. Nor does SEXTANT® allow for any variation in the procedure, if need be.

Depuy Spine's VIPER™ system is another minimally invasive implant and technique recommended for one or two level spine fusions. This system is less complicated than the SEXTANT® only requiring two incisions for a unilateral, one-level fusion, but it is limited in the same way as the SEXTANT® because it also requires the instrumentation to be rigidly fixed to the pedicle screw.

Spinal Concept's PATHFINDER® and NuVasive's SPHERX® spinal system (as disclosed in U.S. Pat. No. 6,802,844), are marketed as “minimally disruptive” spine fusion implants and procedures. While they have advantages over a general “open” procedure, they do not provide all of the advantages of a truly minimally invasive approach. Their characterization as “minimally open” procedures is a result of the inherent difficulty of introducing a rod in a minimally invasive spinal procedure. In order to introduce a rod long enough to accomplish a single level fusion, these systems describe an incision long enough to accept such a rod, thereby undermining the advantages of a minimally invasive approach.

The problem of rod introduction warrants further discussion as it is the central problem in minimally invasive spinal fusions. The systems currently on the market address this issue by adding another incision, using a larger incision, or avoiding fusions greater than one level.

In order to be truly minimally invasive, a spine fusion procedure should have a minimum number of small incisions and not require significant tissue and/or muscle retraction. Furthermore, an improved approach should encompass as many variations and applications as possible thereby allowing the surgeon to adjust the procedure to accommodate the anatomy and surgical needs of the patient as presented. For instance, spinal fusions should not be limited to just one or two levels.

Therefore, a continuing need exists for an improved device, an improved system, and an improved method for performing minimally invasive spine surgery.

### SUMMARY

The present disclosure is directed towards a device, a system, and a method for a screw-based retractor used in performing minimally invasive spine surgery. In some embodiments, the retractor is monolithically formed as part of and has blades that are frangible from a pedicle bone screw that acts as a point of fixation with respect to the patient. The retractor acts as a guide that aids in the insertion of instruments and implants into the anatomy of a patient.

In its nominal position, the retractor has a generally cylindrical configuration with at least one retracting blade. Instrument holes may be located perpendicular to the long axis of each retracting blade whereby a standard surgical instrument, such as a Gelpi Retractor, can be used to spread the blades apart to retract the skin and soft tissue and maintain the field of view and/or working site.

The freedom from obstruction decreases the need for retractor re-positioning during a procedure. In some embodiments, the retractor has a “living hinge” incorporated into the blade. In some embodiments, more than one living hinge can be incorporated into each blade to allow the blade to bend at multiple locations along its length.

As viewed along a longitudinal axis, a cross-section of the retractor has a generally circular configuration that provides additional stiffness. The geometry of the retractor provides sufficient stiffness for maintaining an opening at the surgical site.

An optional window may be located in the blade to allow additional access of instruments into the surgical site.

The distal tip of the minimally invasive retractor is tapered to aid in the insertion of the retractor through the soft tissue. Upon completion of the procedure, the surgeon separates the blades from the pedicle screw tulip and pulls the blades straight out of the wound. The distal end of the retractor may have one or more relief features to aid in the separation of the blades from the pedicle screw tulip.

Multiple retractors may be used during a single spine procedure. The retractor is manufactured for a single use. A method for using the minimally invasive retractor, as disclosed herein, is also disclosed.

### BRIEF DESCRIPTION OF THE DRAWINGS

Embodiments of the presently disclosed minimally invasive retractor are described herein with reference to the accompanying drawings, wherein:

FIG. 1 is a perspective view of a minimally invasive retractor according to a first embodiment of the present disclosure;

FIG. 2 is an enlarged side view of a distal region of the minimally invasive retractor of FIG. 1;

FIG. 3 is a cross-sectional top view of the minimally invasive retractor of FIG. 2 taken along section line 3-3;

FIG. 4 is a perspective view of a minimally invasive retractor according to a further embodiment of the present disclosure;

FIG. 5 is a top view of the minimally invasive retractor of FIG. 4 showing a rod extending through an expanded passage of the minimally invasive retractor;

FIG. 6 is an enlarged side view of a distal region of the minimally invasive retractor of FIG. 4;

FIG. 7 is a cross-sectional top view of the minimally invasive retractor of FIG. 6 taken along section line 7-7; and

FIG. 8 is a cross-sectional side view of the minimally invasive retractor of FIG. 1 in use.

Other features of the present disclosure will become apparent from the following detailed description, taken in conjunction with the accompanying drawings, which illustrate, by way of example, various principles of the present disclosure.

### DETAILED DESCRIPTION OF THE EMBODIMENTS

A device, system, and method for orthopedic spine surgery using a screw based retractor is described in U.S. patent application Ser. No. 11/528,223, filed Sep. 26, 2006, and published on May 10, 2007 as U.S. Published Patent Application Number 2007/0106123, the entire contents of which is incorporated by reference herein. The retractor is designed to be coupled with a pedicle screw and has blades that are frangible from the pedicle screw. A portion of the retractor is removed from the surgical site after use.

Embodiments of the presently disclosed minimally invasive retractor will now be described in detail with reference to the drawings wherein like reference numerals identify similar or identical elements. In the drawings and in the description that follows, the term “proximal,” will refer to the end of a device or system that is closest to the operator, while the term “distal” will refer to the end of the device or system that is farthest from the operator. In addition, the term “cephalad” is used in this application to indicate a direction toward a patient's head, whereas the term “caudad” indicates a direction toward the patient's feet. Further still, for the purposes of this application, the term “medial” indicates a direction toward the middle of the body of the patient, whilst the term “lateral” indicates a direction toward a side of the body of the patient (i.e., away from the middle of the body of the patient). The term “posterior” indicates a direction toward the patient's back, and the term “anterior” indicates a direction toward the patient's front.

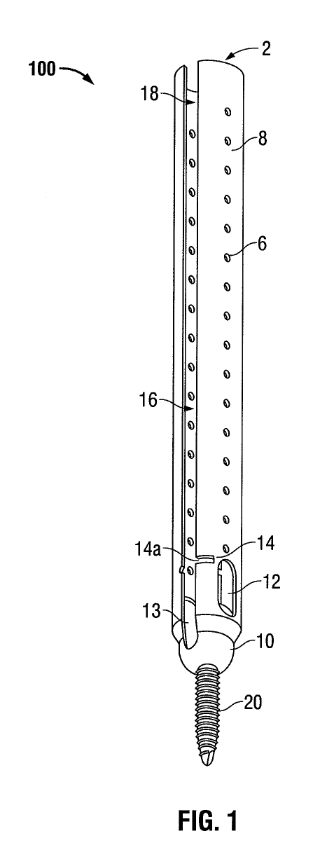

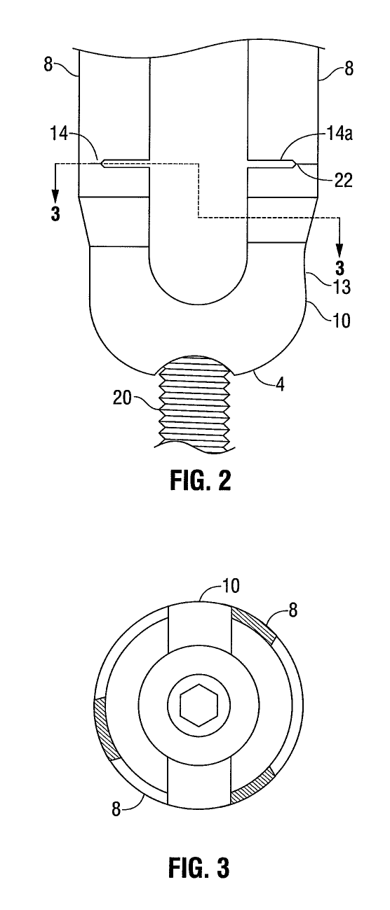

Referring intially to FIGS. 1 and 2, a first embodiment of the presently disclosed minimally invasive retractor or retractor is illustrated and generally designated as 100. Retractor 100 includes an open proximal end 2 and a distal end 4. A longitudinal axis is defined as extending through the center of the proximal end 2 and the distal end 4. The distal end 4 of retractor 100 is a pedicle screw tulip 10 having a generally convex outer surface that facilitates insertion of the retractor 100 through layers of body tissue. A retractor assembly includes a pedicle screw 20 that may be a monoaxial screw, as shown, or a polyaxial screw in combination with retractor 100.

A pair of arms 13 extend distally from the pedicle screw tulip 10. Each of the pair of arms 13 may include at least one slot or window 12. Window 12 may be sized and configured to receive instruments therethrough and/or permit inspection of tissue.

Each of the arms 13 may extend to a flexible region or living hinge 14, defined by the pair of recesses 14a on each side of the living hing 14. As illustrated in FIG. 2, a retrator blade 8 is attached to and extends from the living hinge 14 to define a substantially continuous elongate member. In addition, each retractor blade 8 may have a plurality of instrument holes 6 disposed therethrough. Instrument holes 6 are configured and dimensioned to cooperate with different surgical instruments, such as a Gelpi retractor.

The retractor 100 has a substantially circular cross-section. Each blade 8 and arm 13 has an arcuate cross-sectional configuration that is less than about 180°. A pair of continuous slots 16 separate one blade 8 and arm 13 set from the other blade 8 and arm 13 set.

The pair of continuous slots 16 define a passage 18 that extends substantially the entire length of retractor 100. Passage 18 is expandable for receiving a rod 3 (FIG. 5) therein. Retractor blades 8 and arms 13 define a substantially circular ring shape, thereby providing sufficient stiffness (i.e. rigidity) such that retractor blades 8 and arms 13 resist bending from the counter forces of the retracted tissues and are thinner than the screw housing wall.

It is envisioned that the retractor blades 8 have non-uniform cross-sections. One blade 8 may be semi-circular and the other may be flat. Further, the cross-section of the proximal end 2 of each blade 8 may be circular to allow the retractor 100 to be spread by the insertion of a spreader having arms parallel to the longitudinal axis of the retractor.

Retractor 100 is formed from a suitable biocompatible material that is sterilizable in a suitable configuration and thickness so as to be sufficiently rigid to provide retraction of tissue, and yet is sufficiently bendable to be spread apart to provide retraction and to allow forcible separation of the blades 8 from the pedicle tulip 10 as necessary and appropriate. It is contemplated that retractor 100 be formed from polymers such as polypropylene, polyethylene, or polycarbonate; silicone; polyetheretherketone (“PEEK”); titanium; titanium alloy; surgical steel; or another suitable material including a combination of materials. The blades 8, arms 13, and screw tulip 10 may be formed from the same or different material.

Each retractor blade 8 is capable of being bent away from the centerline of retractor 100, at living hinge 14, in response to applied forces. Bending retractor blade 8 away from the centerline (i.e. radially outwards) increases the width of the passage 18 and acts to retract the surrounding tissue at the selected surgical site.

The retractor blades 8 and the pedicle tulip 10 form a single monolithic structure. The arms 13 are formed or machined to extend proximally from the pedicle tulip 10, as shown in FIGS. 1-3. It is envisioned that a single piece of material be formed and then arms 13 are drilled and machined to create the finished retractor.

It is also envisioned that the arms 13 be co-molded or molded over the pedicle tulip 10. The pedicle tulip 10, including arms 13, is then combined with a screw 20 to form an assembled retractor 100. This configuration allows the force of retraction to be leveraged off of the pedicle screw 20.

The construction of the arms 13 allow the blades 8 to flex outward from the center of the retractor. Repeated movement or flexure of the blades 8 from a radially inward position to a radially outward position, or the use of a separate cutting tool may be used to separate the blades 8 and/or arms 13 from the pedicle tulip 10. A specific layering of composites or the use of different materials in forming the structure can provide a point of separation for the blades 8 and/or arms 13 from the pedicle tulip 10.

To aid in the removal of arms 13 and/or blades 8, a line around the circumference of the retractor 100 can also be etched into the surface to form a scoreline 22. The scoreline 22 (see FIG. 2) allows the arms 13 and/or blades 8 to be separated from the pedicle tulip 10 at a specific location. Scoreline 22 is a physical notch located proximally from the pedicle tulip 10 on the inside, outside, or both inside and outside surfaces. The notch provides a precise location for concentration of fatigue when the arms 13 and/or blades 8 are repeatedly flexed. The fatigue causes separation or breaking at the scoreline 22. The scoreline 22 may be located through the center of a living hinge 14 to facilitate removal of a retractor blade 8 during surgery. Removal of the arms 13 and/or blades 8 from the surgical site is accomplished by separating the arms 13 and/or blades 8 from the pedicle tulip 10 and pulling the blades 8 proximally (i.e. away from the pedicle screw). As such, the physician can readily remove the retractor arms 13 and/or blades 8 from the surgical site while leaving the pedicle tulip 10 and screw 20 in the work area.

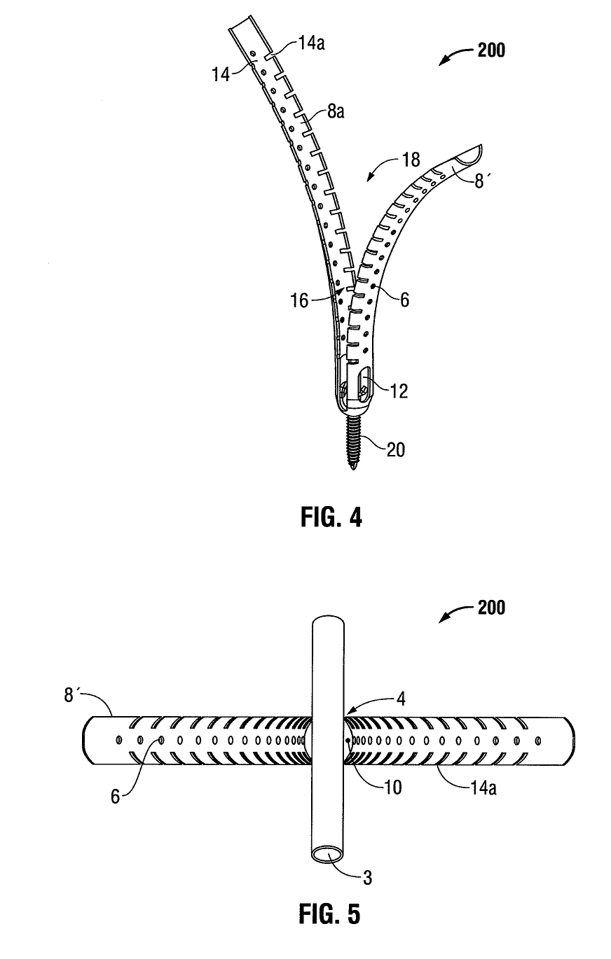

Another embodiment of the presently disclosed retractor is illustrated in FIGS. 4 -7 and shown generally as retractor 200. Retractor 200 is similar to retractor 100, but includes a plurality of living hinges 14 along with their corresponding recesses 14a. Retractor 200 is about 6 inches long and is readily adjusted to a desired length by removing excess material using scissors, a knife, or breaking along a scoreline. Slot 16 is typically at least 5.5 mm wide, but will vary according to the size of the rod 3 inserted into the patient. In particular, each retractor blade 8′ includes a plurality of blade sections 8a. Each blade section 8a is connected to an adjacent blade section 8a by a living hinge 14. The plurality of blade sections 8a and living hinges 14 define retractor blade 8′. As in the previous embodiment (FIG. 1), each blade 8′ and arm 23 set is substantially parallel to anther blade 8′ and arm 23 set to to define a pair of continuous slots 16.

When retractor blades 8′ are urged radially outward from their initial or rest position towards their retracted position, the size of passage 18 increases. This increase in the size and area of passage 18 improves access to the surgical target site (i.e. area where the retractor is inserted into tissue), thereby increasing visibility of the target site, access for instruments, and access for surgical implants. As shown in FIG. 5, rod 3 is positioned in passage 18 after the surrounding tissue has been retracted using retractor 200. Additionally, the plurality of living hinges 14 increases the adaptability of retractor 200 in comparision to retractor 100.

While retractor blades 8 of retractor 100 (FIG. 1) generally bends at its single living hinge 14, the additional living hinges 14 of retractor 200 permit bending with increased flexibility at a number of positions along the length of each retractor blade 8′. As a result, retractor blades 8′ bend at the living hinge 14 that corresponds to a plane defined by the surface of the patient's body tissue. By using this construction, retractor 200 is usable in patient's having different tissue thicknesses between the vertebral body and the surface of their skin. In addition, since each retractor blade 8′ has a plurality of living hinges 14 and blade sections 8a, each retractor blade 8′ can bend at different points along the length of retractor 200. Blades 8′ can accommodate variances in the depth that retractor 200 is inserted. For example, one retractor blade 8′ may bend at its fourth living hinge 14 and the other retractor blade 8′ may bend at its sixth living hinge 14.

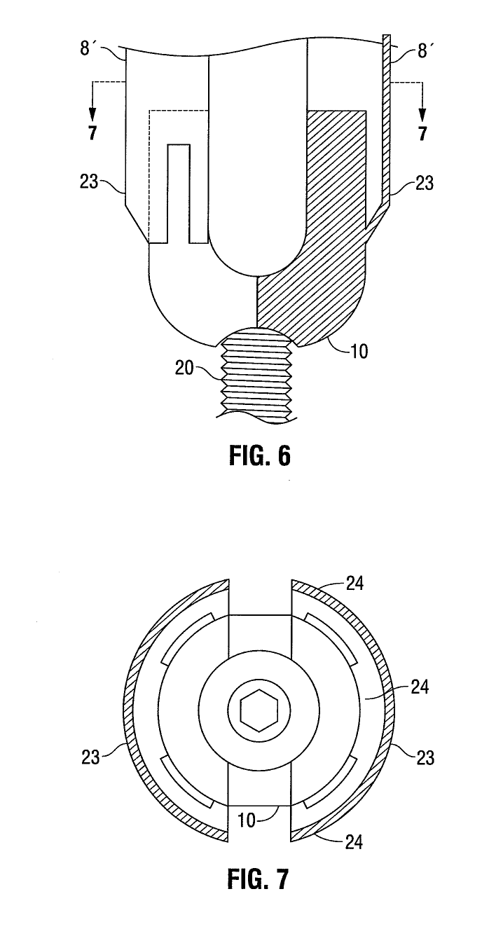

Retractor 200 includes arms 23, formed to project radially outward from the sides of the pedicle tulip 10 via attachments 24 before extending proximally, as shown in FIGS. 6 and 7. This construction allows for a separate tool to be placed between the arms 23 and the tulip 10 for removal of the arms 23 at the attachment points.

In another embodiment, minimally invasive retractor may be constructed to include two blades integrally formed and attached on only one side of the tulip, thereby increasing the lateral opening near the pedicle tulip to define a window that is larger than the previously disclosed window 12 of retractor 100. This embodiment provides increased access to the target site and allows larger implants or instruments to be positioned within the target site.

The presently disclosed retractor may also include only one retractor blade allowing greater variability in creating the retracted space, as well, increased access to the target site for using larger instruments or inserting larger devices than possible with retractors 100 or 200.

It is contemplated that any of the previously disclosed retractors may be formed of a resilient material. When external spreading forces (i.e. from a Gelpi retractor or the physician's hands) are removed, the retractor blades return towards their initial position of being substantially parallel to the centerline. It is also contemplated that any of the previously disclosed retractors may be formed of a bendable non-resilient material that resists returning to their initial position and remain in the retracted position.

The presently disclosed retractors utilize, but are not limited to, a method whereby an initial incision is made in the skin of approximately 10-15 mm in length. Surgeon preference will dictate the need for one or more stages of dilators to aid in expanding the wound before introducing one or more retractors. Normal surgical techniques may be used to close the incision(s).

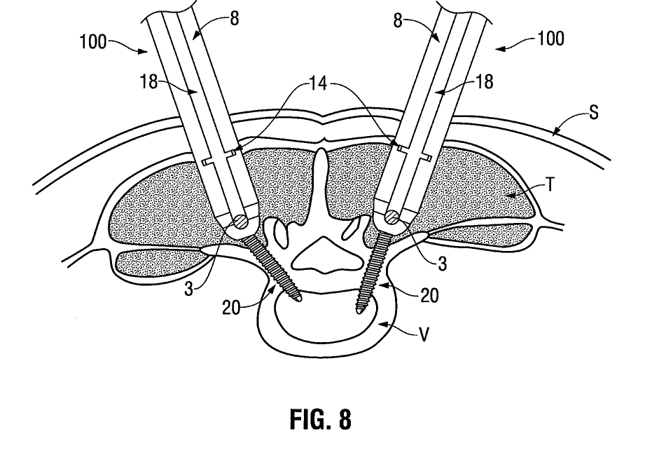

A method for use of the presently disclosed system will now be described with reference to FIG. 8. The retractor is inserted into an incision through the patient's skin S and muscle/fat tissue T such that pedicle screw 20 is subsequently threaded into a vertebral body V. Once the desired number of retractors 100 are affixed to vertebral bodies V, retractor blades 8 are spread and/or pivoted apart to retract skin S and tissue T to create a retracted area at the target site. Rod 3 is inserted in pasage 18 when passage 18 is in an expanded state (i.e., tissue has been retracted).

The rod may be inserted along a path from one screw head to another, possibly subcutaneously to be secured to fastening regions of pedicle screws in adjacent vertebral bodies. The retractors of the present disclosure are well suited for such a technique due to the unique access provided. Once the screw-rod construct is complete, the retractor blades 8 and/or arms 13 are separated as described above, from the screw tulip 10, and then pulled out of the incision.

Removal may be done by hand or with suitable gripping tools. An example of a retractor extracting tool is described in U.S. Published Patent Application Number 2007/0106123 (referenced hereinabove). The blades 8 and/or arms 13 are separated from pedicle tulip 10 without imparting significant downward or rotational forces against the patient's body. The blade 8 and/or arms 13 may then be removed from the patient and this process may be repeated for each installed retractor 100.

The retractor may be manufactured from medical grade plastic or metal, thermoplastics, composites of plastic and metal, or biocompatible materials. The plastic retractor may be made from, but not limited to, polypropylene and polyethylene. The plastic retractor may be transparent or opaque and may have radio opaque markers for visibility during various imaging techniques. The metallic retractor utilizes such materials as, but not limited to, aluminum, stainless steel, and titanium. The retractor may have a nonconducting outer coating. In addition, the parts may have a reflective or non-reflective coating to increase visibility in the surgical site and may have an artificial lighting feature.

As with any surgical instrument and implant, the retractors must have the ability to be sterilized using known materials and techniques. Parts may be sterile packed by the manufacturer or sterilized on site by the user. Sterile packed parts may be individually packed or packed in any desirable quantity. For example, a sterile package may contain one or more retractors in a sterile enclosure. Alternatively, such a sterile surgical kit may also include one or more bone biopsy needles, Jamshidi needle(s), guide wires, sterile cannulated scalpels, dilators, rods, or other surgical instruments and combinations thereof as contemplated in in U.S. patent application Ser. No. 12/104653, filed on Apr. 17, 2008, and published on Oct. 23, 2008 as U.S. Published Patent Application Number 2008/0262318, the entire contents of which are hereby incorporated by reference.

The blades may be made of a light transmitting material. The retractor may include a light guide system. The light guide system has an input adapter to receive light from a light source and one or more light emitting surfaces to illuminate the surgical field.

It will be understood that various modifications may be made to the embodiments of the presently disclosed retraction system. Therefore, the above description should not be construed as limiting, but merely as exemplifications of embodiments. Those skilled in the art will envision other modifications within the scope and spirit of the present disclosure.

For example, while the foregoing description has focused on spine surgery, it is contemplated that the retractors and methods described herein may find use in other orthopedic surgery applications, such as trauma surgery. The present disclosure may be used, with or without a bone screw to insert a screw or pin into bone in a minimally invasive manner, or otherwise to access a surgical target site over a guidewire, dilator, scalpel, or retractor.

Further still, it will be appreciated that the pedicle screw may be cannulated to allow it to be translated along a guide wire to facilitate percutaneous insertion of the pedicle screw and retractor. In addition, it is contemplated that conventional insertion tools or those disclosed in U.S. Published Patent Application Number 2008/0262318 (referenced hereinabove), the entire contents of which are hereby incorporated by reference, be used in conjunction with the presently disclosed retractor and pedicle screws.

1. A surgical retractor comprising:
a pedicle screw having a tulip; and
at least one elongate member formed integrally with and extending from said tulip, the elongate member having a flexible joint region and being removably affixed to the tulip.

2. The retractor of claim 1, wherein the flexible joint region includes first and second arcuate arms extending proximally from the distal end, the arcuate arms being radially spaced apart and defining a pair of longitudinally extending slots.

3. The retractor of claim 1, further comprising at least two elongate arcuate members, the elongate arcuate members being radially spaced apart and defining a pair of longitudinally extending passages.

4. The retractor of claim 3, further comprising at least two elongate arcuate members, the elongate arcuate members being radially spaced apart and defining a pair of longitudinally extending passages, the pair of longitudinally extending passages being substantially aligned with the longitudinally extending slots.

5. The retractor of claim 1, further comprising at least two elongate arcuate members, each of the elongate arcuate members having a plurality of holes located perpendicular to the long axis of each elongate arcuate member configured to accept a standard surgical instrument used to spread the blades apart.

6. A method of performing spine surgery comprising the steps of:
a) providing at least two retractor assemblies, each retractor assembly including at least one elongate member flexibly and frangibly attached to a pedicle screw;
b) securing the first screw to a portion of a first vertebral body;
c) retracting tissue using the at least one elongate member of the first retractor;
d) securing the second screw to a portion of a second vertebral body;
e) retracting tissue using the at least one elongate member of the second retractor;
f) inserting a rod between the first and second screws;
g) securing the rod to the first and second screws: and
h) removing the elongate members from the pedicle screws.

7. A method of performing surgery comprising:
a) percutaneously inserting a guidewire into a boney structure;
b) inserting a cannulated bone screw with an integrally formed, frangible retractor member over the guidewire to the target site;
c) implanting the bone screw into bone;
d) spreading the retractor from a pivot point adjacent the bone screw to retract soft tissue and provide access to the site;
e) performing a surgical procedure at the target site through the retractor;
f) removing the integrally formed retractor member from the bone screw.

8. A kit comprising a sterile enclosure containing:
a surgical pedicle screw having at least one frangible elongate retracting member, and at least one relief region, and a flexible joint coupling the elongate member to the pedicle screw.

9. The kit of claim 8, further comprising a plurality of surgical retractors disposed within the sterile enclosure.

10. The kit of claim 9, further comprising a cannulated scalpel disposed within the sterile enclosure.

11. The kit of claim 9, further comprising at least one dilator disposed within the sterile enclosure.

12. The kit of claim 9, further comprising at least one biopsy needle disposed within the sterile enclosure.

13. The kit of claim 9, further comprising at least one length of a guidewire disposed within the sterile enclosure.
